(19) United States
(12) Patent Application Publication
(10) Pub. No.: US 2020/0000391 A1
(43) Pub. Date: Jan. 2, 2020

(54) OPERATION APTITUDE JUDGMENT DEVICE AND OPERATION APTITUDE JUDGMENT METHOD

(71) Applicant: MITSUBISHI ELECTRIC CORPORATION, Tokyo (JP)

(72) Inventor: Jumpei HATO, Tokyo (JP)

(73) Assignee: MITSUBISHI ELECTRIC CORPORATION, Tokyo (JP)

(21) Appl. No.: 16/469,315

(22) PCT Filed: Mar. 3, 2017
(86) PCT No.: PCT/JP2017/008591

§ 371 Date: Jun. 13, 2019

(51) Int. Cl. A61B 5/18; B60R 21/015; G06K 9/00; B60W 40/08; G06F 3/01; G08G 1/16

(52) U.S. Cl. CPC A61B 5/18; B60R 21/01552; G06K 9/00845; B60W 40/08; B60W 2040/0863; G08G 1/163; G06K 9/00664; G06K 9/00805; G06F 3/013

(57) ABSTRACT

An operation aptitude judgment device includes a perception difficulty space detection unit that detects a perception difficulty space in which a perception object as an object that the user should perceive when the user performs a planned operation is difficult for the user to perceive based on vicinal object information acquired from a vicinal object detection device that detects a vicinal object existing in a vicinity of the user; a user perception movement detection unit that detects a user perception movement, as a movement of the user when the user tries to perceive the perception object, based on user movement information acquired from a user movement detection device that detects a movement of the user; and an operation aptitude level calculation unit that calculates the operation aptitude level of the user based on the perception difficulty space and the user perception movement.

### TECHNICAL FIELD

The present invention relates to an operation aptitude judgment device, an operation aptitude judgment method and an operation aptitude judgment program for judging an operation aptitude level indicating in how suitable condition a user is to perform an operation that should be carried out.

### BACKGROUND ART

Conventionally, various technologies have been proposed for judging in how suitable condition a driver as a user (operator) of an automobile is for the driving of the automobile as an operation that should be carried out.

For example, Non-patent Reference 1 proposes a system that uses a smartphone-dedicated application equipped with a sleepiness detection algorithm and a wearable heart rate meter for measuring the heart rate of a driver, detects sleepiness of the driver based on the heart rate, and issues a warning to the driver while e-mailing a warning to a manager of the driver.

Patent Reference 1 proposes a technology for determining an object that should be visually recognized, detecting whether a driver has visually recognized the object that should be visually recognized or not based on the driver's line of sight detected based on a face image of the driver, and judging an operation aptitude level of the driver. Here, the object that should be visually recognized is, for example, a traffic sign, a traffic signal, a vehicle, an obstacle or a moving object such as a pedestrian.

### PRIOR ART REFERENCE

### Non-Patent Reference

Non-patent Reference 1: NTT Data MSE Corporation, Kyoto University, Kumamoto University, NTT DoCoMo, Inc., Press Release “Demonstration Experiment Started for Sleepiness Detection System for Drivers Utilizing hitoe” [online], May 10, 2016, Internet, URL: https://www.nttdocomo.co.jp/info/news_release/2016/05/10_00.html
Non-patent Reference 2: Keisuke Morishima and five others, “Measurement of Useful Field of View in Eye and Head-Free Condition while Driving”, Transactions of the Japan Society of Mechanical Engineers (Part C), October 2013, Vol. 79, No. 806, pp. 272-284 (pp. 3561-3573)

### Patent Reference

Patent Reference 1: Japanese Patent Application Publication No. 2009-69885

### SUMMARY OF THE INVENTION

### Problem to be Solved by the Invention

However, in the technology proposed by the Non-patent Reference 1, the driver has to take care not to forget to wear the wearable heart rate meter, and the driver can find it troublesome to wear the wearable heart rate meter or find the wearable heart rate meter bothersome after wearing the wearable heart rate meter. Thus, there is a problem of imposing a burden on the driver.

The technology proposed by the Patent Reference 1 has the following problem:

In general, a user as an operator carries out a planned operation by repeating activity including:

(Action 1) collecting information necessary for appropriately carrying out the planned operation from the surrounding environment or the like (i.e., recognizing necessary information),

(Action 2) considering starting what type of movement makes it possible to appropriately carry out the operation based on the collected information (i.e., judging), and

(Action 3) putting the operation into practice (i.e., controlling action) according to the contents of the consideration (i.e., result of the judgment).

Therefore, it is possible to judge that the user is capable of appropriately carrying out the operation if the user is in a condition of being capable of appropriately performing (Action 1) to (Action 3).

In the method employing the “recognizing necessary information” indicated in (Action 1) as a criterion of judgment (referred to as a “recognition-based aptitude judgment method”), it is necessary to confirm that the user has recognized the necessary information. However, the recognition is internal activity of the user and measurement of the recognition is difficult. For example, even if behavior of a sensory organ of the user is observed, it is difficult to precisely distinguish whether the behavior of the sensory organ is a result of a reflexively reacting to a perception object, that is, an object that should be perceived (i.e., reflexive action that has not reached recognition) or a result obtained based on recognition of the perception object (i.e., an action performed based on recognition). Therefore, it is difficult to precisely distinguish whether movement of the line of sight, as the user's behavior employed in the technology described in the Patent Reference 1, is a reflexive action due to high remarkableness of the perception object at the end of the line of sight or an action performed based on recognition. Thus, there is a problem in that the operation aptitude level cannot be judged precisely.

An object of the present invention, which has been made to resolve the above-described problems, is to provide an operation aptitude judgment device and an operation aptitude judgment method with which the operation aptitude level indicating in how suitable condition the user is to perform a planned operation can be judged precisely without imposing a burden on the user, and to provide an operation aptitude judgment program that makes it possible to execute the operation aptitude judgment method.

### Means for Solving the Problem

An operation aptitude judgment device according to an aspect of the present invention is a device that judges an operation aptitude level indicating in how suitable condition a user is to perform a planned operation that should be carried out, including:

a perception difficulty space detection unit that detects a perception difficulty space in which a perception object as an object that the user should perceive when the user perfoims the planned operation is difficult for the user to perceive based on vicinal object information acquired from a vicinal object detection device that detects a vicinal object existing in a vicinity of the user; a user perception movement detection unit that detects a user perception movement, as a movement of the user when the user tries to perceive the perception object, based on user movement information acquired from a user movement detection device that detects a movement of the user; and an operation aptitude level calculation unit that calculates the operation aptitude level of the user based on the perception difficulty space detected by the perception difficulty space detection unit and the user perception movement detected by the user perception movement detection unit.

An operation aptitude judgment method according to another aspect of the present invention is a method of judging an operation aptitude level indicating in how suitable condition a user is to perform a planned operation that should be carried out, the method including: detecting a perception difficulty space in which a perception object as an object that the user should perceive when the user performs the planned operation is difficult for the user to perceive based on vicinal object information acquired from a vicinal object detection device that detects a vicinal object existing in a vicinity of the user; detecting a user perception movement, as a movement of the user when the user tries to perceive the perception object, based on user movement information acquired from a user movement detection device that detects a movement of the user; and calculating the operation aptitude level of the user based on the detected perception difficulty space and the detected user perception movement.

### Effect of the Invention

According to the present invention, an advantage is obtained in that the operation aptitude level indicating in how suitable condition the user is to perform an operation can be judged precisely without imposing a burden on the user.

### BRIEF DESCRIPTION OF THE DRAWINGS

FIG. 1 is a block diagram schematically showing a configuration of an operation aptitude judgment device according to first and second embodiments of the present invention.

FIG. 2 is a diagram schematically showing a hardware configuration of the operation aptitude judgment device according to the first and second embodiments.

FIG. 3 is a diagram showing an example of data collected by a vicinal object detection device.

FIG. 4 is a diagram showing another example of data collected by the vicinal object detection device.

FIG. 5 is a sequence diagram showing a basic process executed by the operation aptitude judgment device according to the first and second embodiments.

FIG. 6 is a sequence diagram showing details of an internal process of a main loop process in the operation aptitude judgment device according to the first embodiment.

FIG. 7 is a diagram showing a concrete perception difficulty space detection process in regard to perception by means of the sense of sight.

FIG. 8 is a diagram showing an example of a method of judging importance of a perception difficulty space.

FIG. 9 is a diagram showing another example of the method of judging the importance of the perception difficulty space.

FIG. 10 is a diagram showing a situation in which there exists a perception difficulty space caused by a vicinal object in regard to a viewpoint position of a user as a reference point.

FIG. 11 is a diagram showing an example of a perception importance level in regard to each position on a plane surface including a line segment passing through two points on the vicinal object.

FIG. 12 is a diagram showing a situation in which the vicinal object in FIG. 10 is another vehicle and there exists a perception difficulty space caused by the vicinal object in regard to the viewpoint position of the user as the reference point.

FIG. 13 is a diagram showing an example of the perception importance level in regard to each position on the plane surface including the line segment passing through two points on the vicinal object in the situation of FIG. 12.

FIG. 14 is a sequence diagram showing details of an internal process of a main loop process in an operation aptitude judgment device according to the second embodiment.

FIG. 15 is a diagram for explaining a perception object detection process in FIG. 14.

FIG. 16 is a diagram for explaining a user perception object judgment process in FIG. 14.

### MODE FOR CARRYING OUT THE INVENTION

Operation aptitude judgment devices, operation aptitude judgment methods and operation aptitude judgment programs according to embodiments of the present invention will be described below with reference to the accompanying drawings. In first and second embodiments, the description will be given mainly of cases where the operation is a driving of an automobile and a user performing the operation is a driver of the automobile. However, the following embodiments are just examples and a variety of modifications are possible within the scope of the present invention.

### (1) First Embodiment

### (1-1) General Outline

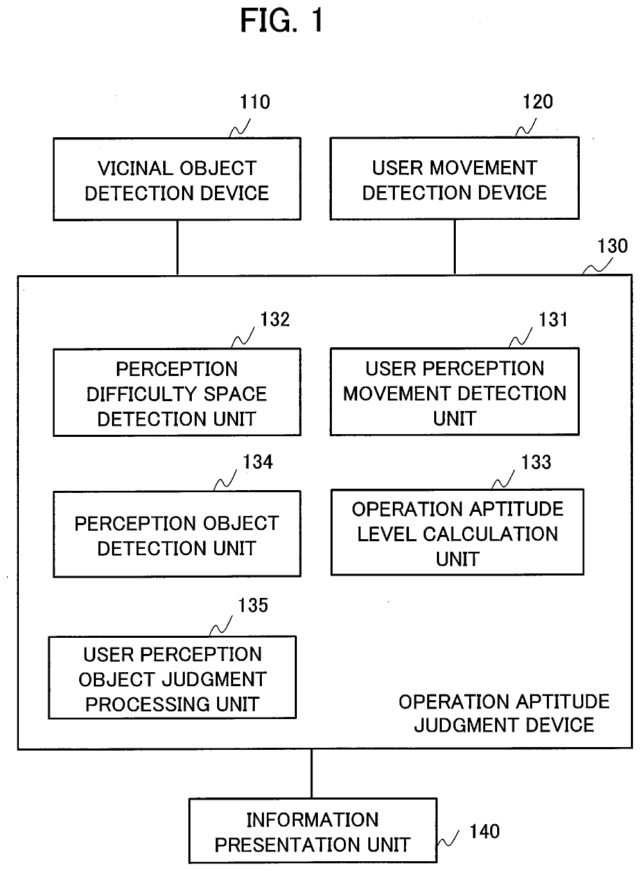

FIG. 1 schematically shows a configuration of an operation aptitude judgment device 130 according to the first embodiment. The operation aptitude judgment device 130 is a device capable of executing an operation aptitude judgment method according to the first embodiment. The operation aptitude judgment method can be executed by an operation aptitude judgment program as software stored in the operation aptitude judgment device or a server.

The operation aptitude judgment device 130 is a device that judges an operation aptitude level indicating in how suitable condition the user is to perform a planned operation that should be carried out. The operation aptitude judgment device 130 acquires vicinal object information obtained by detecting one or more objects in the vicinity of the user (in a surrounding area of or around the user) from a vicinal object detection device 110, and acquires user movement information obtained by detecting movement of the user from a user movement detection device 120. The operation aptitude judgment device 130 calculates the operation aptitude level of the user by using the acquired vicinal object information and user movement information and provides an information presentation unit 140 with the calculated operation aptitude level. The information presentation unit 140 is capable of informing the user of how suitable or how unsuitable the present condition is to perform the planned operation.

As shown in FIG. 1, the operation aptitude judgment device 130 includes a user perception movement detection unit 131, a perception difficulty space detection unit 132 and an operation aptitude level calculation unit 133. The perception difficulty space detection unit 132 detects a perception difficulty space in which a perception object as an object that the user should perceive when the user performs a planned operation is difficult for the user to perceive by using the vicinal object information acquired from the vicinal object detection device 110. The user perception movement detection unit 131 detects a user perception movement, as a movement of the user when the user tries to perceive the perception object, by using the user movement information acquired from the user movement detection device 120. The operation aptitude level calculation unit 133 calculates the operation aptitude level of the user based on the perception difficulty space detected by the perception difficulty space detection unit 132 and the user perception movement detected by the user perception movement detection unit 131.

As above, the first embodiment takes advantage of the fact that the perception difficulty space is not an object having high remarkableness differently from perception objects. Specifically, when there exists a perception difficulty space, the user's movement when the user tries to perceive the perception difficulty space, that is, the user perception movement regarding the perception difficulty space, has a high possibility of not being a reflexive action due to high remarkableness of a perception object but being an action performed based on recognition of the perception difficulty space. In other words, according to the first embodiment, the user perception movement is detected when the aforementioned (Action 1) described in the background art is an action performed based on recognition (i.e., not a reflexive action). Thus, with the operation aptitude judgment device 130 according to the first embodiment, the operation aptitude level can be judged precisely and reliability of the operation aptitude level can be increased.

Further, in order to further increase the reliability of the operation aptitude level, the operation aptitude judgment device 130 may further include a user perception object judgment processing unit 135 and a perception object detection unit 134 that detects a perception object by using the vicinal object information acquired from the vicinal object detection device 110. In the first embodiment, a configuration a configuration including neither the perception object detection unit 134 nor the user perception object judgment processing unit 135 will be described. A configuration including the perception object detection unit 134 and the user perception object judgment processing unit 135 will be described in the second embodiment.

### (1-2) Configuration

### Operation Aptitude Judgment

The operation aptitude judgment device 130 according to the first embodiment is a device capable of judging (calculating) the operation aptitude level regarding the user as the driver performing driving of an automobile (vehicle) as the operation. In the operation aptitude judgment, the following processes are performed:

(First Process) A process of detecting the perception difficulty space as a space in which a perception object that the user should perceive when the user performs a planned operation is difficult for the user to perceive (perception difficulty space detection operation).

(Second Process) A process of detecting a user perception movement that is a user's attempt to perceive a perception object (user perception movement detection operation).

(Third Process) A process of calculating the operation aptitude level indicating how suitable the user is to perform the planned operation (i.e., the level of aptitude) by using the detected perception difficulty space and the detected user perception movement (operation aptitude level calculation operation).

### (Perception Object)

The perception objects as vicinal objects that can be perceived by the user during driving (i.e., perceivable objects) can be various objects, and can include, for example, a mobile object such as a vicinal vehicle, a bicycle, a motorcycle, a pedestrian or an animal, a road component such as a roadside strip, a white line, a pedestrian crossing, a median, a traffic sign or a traffic signal, and a fixed object such as a building, a roadside tree or a signboard. The user intermittently repeats moving the line of sight in order to check the condition of a perception object that is judged to be important at the appropriate times. In this case, the user acquires necessary information from the perception object by directly viewing the perception object.

### (User Perception Movement)

The user perception movement means any type of movement of the user trying to acquire information necessary for performing an operation through the five senses. For example, user perception movements by means of the sense of sight include the user's eye movement, the user's line of sight (direction and movement of the line of sight), the user's carefully watching position (range), and so forth. Further, the user perception movements by means of the sense of sight also include the range of an effective visual field estimated from movement of a sensory organ itself, the range of a peripheral visual field as a visual field around the effective visual field, a change in the range of the effective visual field or the peripheral visual field, and so forth. User perception movements by means of the sense of hearing include, for example, movement of assuming a posture suitable for collecting sound around the user such as movements of directing ears in the direction of the source of sound and movement of cupping hands behind the ears. Other user perception movements include a movement for enhancing perceptual sensitivity and a movement for reducing needless movement. For example, the user perception movements also include macro movements such as an action of blocking sensory organs other than a sensory organ whose perceptual sensitivity is desired to be enhanced, like closing eyes or covering ears, and an action of bringing a sensory organ whose perceptual sensitivity is desired to be enhanced close to the object, like bringing the face or ears close to the object by turning round or changing the posture.

Various methods have been developed for the detection of the user's line of sight or the user's carefully watching position. For example, as such detection methods, there have been known a method of detection based on the positional relationship between the inner corner of an eye and the iris of the eye, a method of detection based on the relationship between the position of the pupil and the position of infrared ray cornea reflection occurring when an infrared ray emitted from an infrared LED (Light Emitting Diode) is applied to the user's eye, and so forth. The range of the effective visual field or the like can be measured by the staircase method or the Probit method, or can also be measured by the method described in the Non-patent Reference 2. User perception movements accompanied by the user's macro movements can be detected by using technology in the field collectively referred to as activity recognition.

### (Perception Difficulty Space)

Since the real world is a three-dimensional space, the perception object that should be perceived and that is important in the operation (i.e., object that should be perceived) is not necessarily in a perceivable condition. Specifically, there are cases where the perception object that should be perceived exists at a position hidden behind a certain object and invisible from the user. For example, the perception object that should be perceived can be a child or the like who is about to run out onto the road from behind a vehicle parked on the roadside. There are also situations in which the perception object that should be perceived is not totally hidden behind an object. In such cases, the perception object that should be perceived can be a child whose body parts other than the top of the head are hidden behind a vehicle parked on the roadside, a bicycle that can be visually recognized only through a gap between roadside trees, or the like. As described above, a range in which the user is totally incapable of perceiving the perception object that should be perceived (a range in which even partial perception is impossible) or a range in which partial perception is possible (but a part of the range cannot be perceived), or a range including both of these ranges, is defined as the perception difficulty space. The perception difficulty space regarding the sense of sight means a space generally called a dead space. Anticipating the existence of a perception object hidden in the perception difficulty space and properly directing attention towards the perception object that can emerge from the perception difficulty space is a user action essential for appropriately carrying out a lot of operations.

### (Counter-obstruction Perception Movement)

In general, when the user tries to recognize a risk existing in the perception difficulty space and perceive a perception object hiding in the perception difficulty space, the user performs a user perception movement different from normal user perception movements in order to improve the perception in the present state against a perception obstruction as a factor causing the perception difficulty space.

The normal user perception movement means to direct attention of the obstructed sensory organ towards the perception difficulty space caused by the perception obstruction. A concrete example is a user perception movement of directing the line of sight towards a dead space when there exists the dead space as the perception difficulty space caused by an obstacle and the user worries about something beyond the dead space (spatial part behind the obstacle). In contrast, in order to improve the perception of something beyond the dead space (spatial part behind the obstacle) in the present state, there can occur a user perception movement accompanied by a body motion such as changing the direction of the face, changing the posture, honing the vision, or moving the obstacle causing the dead space if possible. Conversely, there are also cases where a user perception movement accompanied by a decrease in a body motion occurs due to concentration of attention to a particular sensory organ.

Besides the above-described cases, there are cases where a decrease in perceptual sensitivity of a sensory organ occurs, such as a case where concentration of visual attention to a certain dead space leads to a late or no visual reaction to another object or dead space. In regard to the sense or sight, this corresponds to the narrowing of the effective visual field or the peripheral visual field. Such a decrease in the perceptual sensitivity of a sensory organ can occur not only to the sensory organ of the obstructed sensory perception but also to another sensory organ. For example, there are cases where concentration of visual attention to a dead space leads to a decrease in reaction to sound, that is, a decrease in perceptual sensitivity of the sense of hearing.

The above-described characteristic user perception movement such as a body motion appearing as a result of a user's positive attempt to perceive the perception difficulty space, a decrease in the perceptual sensitivity of a sensory organ for an object other than the present perception object, or the like will be referred to as a counter-obstruction perception movement.

### (System Configuration of Operation Aptitude Judgment Device 130)

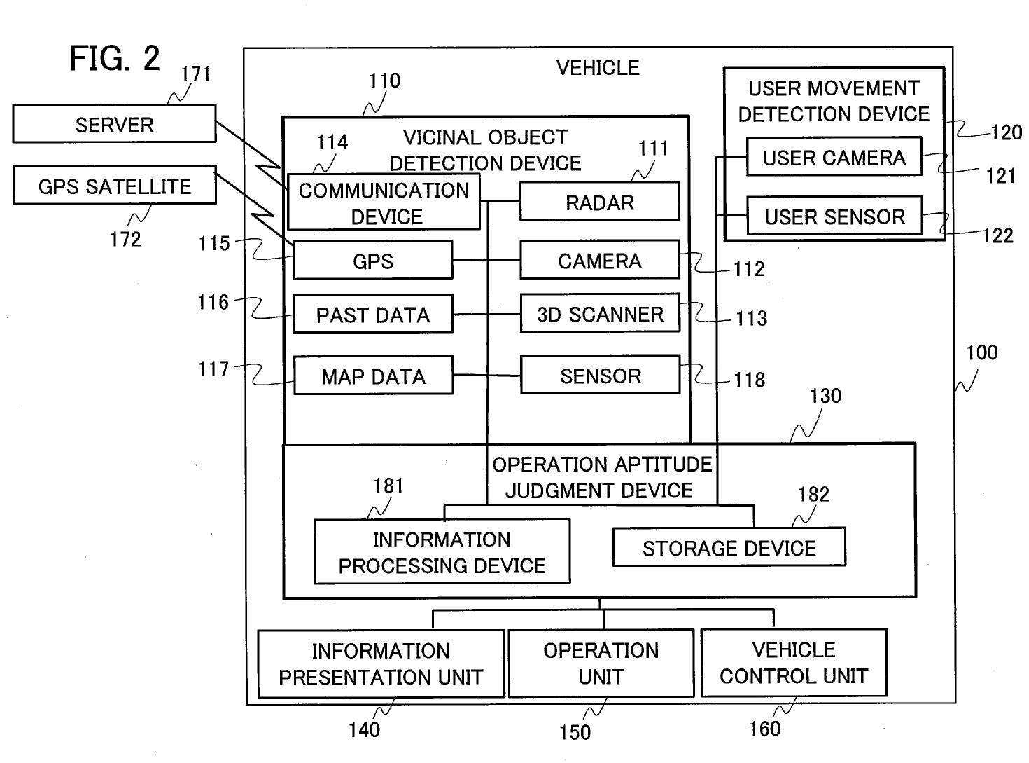

FIG. 2 schematically shows a hardware configuration of the operation aptitude judgment device 130 according to the first embodiment. FIG. 2 shows the operation aptitude judgment device 130 installed in a vehicle 100. As shown in FIG. 2, the vehicle 100 includes the vicinal object detection device 110, the user operation detection device 120, the operation aptitude judgment device 130, the information presentation unit 140, an operation unit 150 and a vehicle control unit 160.

In the example of FIG. 2, the user's driving of the vehicle 100 equipped with the operation aptitude judgment device 130 will be referred to as an “operation”, and the condition of the user being capable of carrying out the operation with no accident will be referred to as a “condition suitable for performing the operation”, that is, a condition at a high operation aptitude level. In general, it is said that approximately 80% of information necessary for driving is acquired through the sense of sight. In the first embodiment, the description will be given mainly of a case where the user perception movement is a movement by means of the sense of sight for the simplicity of the description. However, the present invention is not limited to the sense of sight and the operation aptitude judgment is possible even by use of a sense other than the sense of sight.

Further, while the user in the first embodiment is assumed to be a driver as a vehicle user who drives the vehicle 100, the user in the present invention is not limited to a driver; there are cases, for example, where a passenger seated on the passenger seat or the rear seat who does not drive the vehicle in normal times but drives the vehicle as a substitute driver in exceptional situations, is included in the user. Furthermore, in cases where the vehicle 100 is an autonomous vehicle, the passenger seated on the driver's seat is not the driver; however, the passenger seated on the driver's seat is included in the user since there are cases where the passenger performs part of driving operation.

### (Vicinal Object Detection Device 110)

The vicinal object detection device 110 shown in FIG. 2 includes various devices for collecting data necessary for detecting an object existing in the vicinity of the vehicle 100 (e.g., in the vicinity of the front area of the vehicle 100 in regard to the traveling direction). A radar 111 measures the distance or direction of an object existing in the vicinity of the vehicle 100 by emitting a radio wave to the vicinity of the vehicle and measuring reflected waves at that time. A camera 112 acquires image information by measuring light emitted (reflected) from the vicinity of the vehicle 100 and thereby photographing the vicinity of the vehicle 100. A three-dimensional (3D) scanner 113 measures the distance or direction of an object existing in the vicinity of the vehicle 100 by emitting laser light or the like to the vicinity of the vehicle 100 and measuring reflected light of the emitted light. A sensor 118 includes various types of sensors for detecting various types of signals emitted from objects existing in the vicinity of the vehicle 100. The sensor 118 can include, for example, a microphone for collecting sound, a contact sensor for measuring a contact condition, a temperature sensor for collecting temperature data regarding the vicinity, an infrared thermography, and so forth.

While the vehicle 100 does not necessarily have to be equipped with all of the radar 111, the camera 112, the 3D scanner 113 and the sensor 118, the vehicle 100 is equipped with detectors suitable for detecting an object existing in the vicinity.

Further, while the radar 111, the camera 112, the 3D scanner 113 and the sensor 118 in the first embodiment are assumed to be used for detecting an object existing in the vicinity of the vehicle 100, their measurement ranges are not limited to the vicinity of the vehicle; the inside of the vehicle 100 may also be regarded as the object of measurement in cases where information regarding the vicinity of the user, e.g., the inside of the vehicle 100, also has to be handled as the vicinal object, for example.

A communication device 114 communicates with a server 171 via a network and is used for acquiring data necessary for detecting an object existing outside the vehicle 100 or additional data such as the type and attribute of the detected object or the like. The communication device 114 may be used also for transmitting data obtained by the measurement by the radar 111, the camera 112, the 3D scanner 113, the sensor 118, etc. to the server 171, requesting the server 171 to perform a process such as an object detection process or an additional data search process regarding the type and attribute of the detected object or the like, and receiving the result of the process. The server 171 is not limited to a server machine as a computer (information processing device) for providing service or functions; the server 171 is not particularly limited as long as the server 171 is a device capable of communicating with the communication device 114 and storing data or a device equipped with an information processing device. The server 171 can also be an information processing device mounted on a vicinal vehicle, or another information processing device, for example.

A GPS (Global Positioning System) 115 is used for learning the present position of the vehicle 100 by receiving signals from GPS satellites 172. The present position is transmitted from the communication device 114 to the server 171 and is usable for acquiring information regarding highly perpetual objects existing in the vicinity of the present position, such as buildings, signs and roads.

Map data 117 is stored in a storage device of the vehicle 100 or provided from the server 171 and is used for extracting map data of the vicinity of the present position by using the present position as a key. The map data 117 is data obtained by digitizing geographical condition of part or the whole of the earth surface, and is usable mainly as one of information sources regarding highly perpetual objects existing in the vicinity such as buildings, signs and roads.

Past data 116 is stored in a storage device of the vehicle 100 or provided from the server 171 and can include data regarding objects detected when the vehicle 100 traveled in the past, output data from the radar 111, the camera 112, the 3D scanner 113 and the sensor 118, and so forth. Data regarding highly perpetual objects such as buildings, signs and roads among the objects detected in the past may be recorded together with position data, by which the processing load for detecting objects outside the vehicle can be reduced. The same advantage can be achieved in regard to the output data by recording the output data together with the position data.

The radar 111, the camera 112, the 3D scanner 113 and the sensor 118 are used mainly for detecting objects in the vicinity by measuring the vicinity of the vehicle 100 in real time and for measuring conditions of movement of mobile objects such as vicinal vehicles, pedestrians and bicycles or the like. In contrast, the communication device 114, the past data 116 and the map data 117 are information sources providing data generated based on the result of past measurement and are used for detecting buildings, signs, roads, etc. that are highly perpetual. However, the server 171 with which the communication device 114 communicates can be a mobile object measured by a vehicle in the vicinity of the vehicle 100. In this case, data transmitted from the vehicle in the vicinity can be received in real time.

### (Concrete Examples of Data)

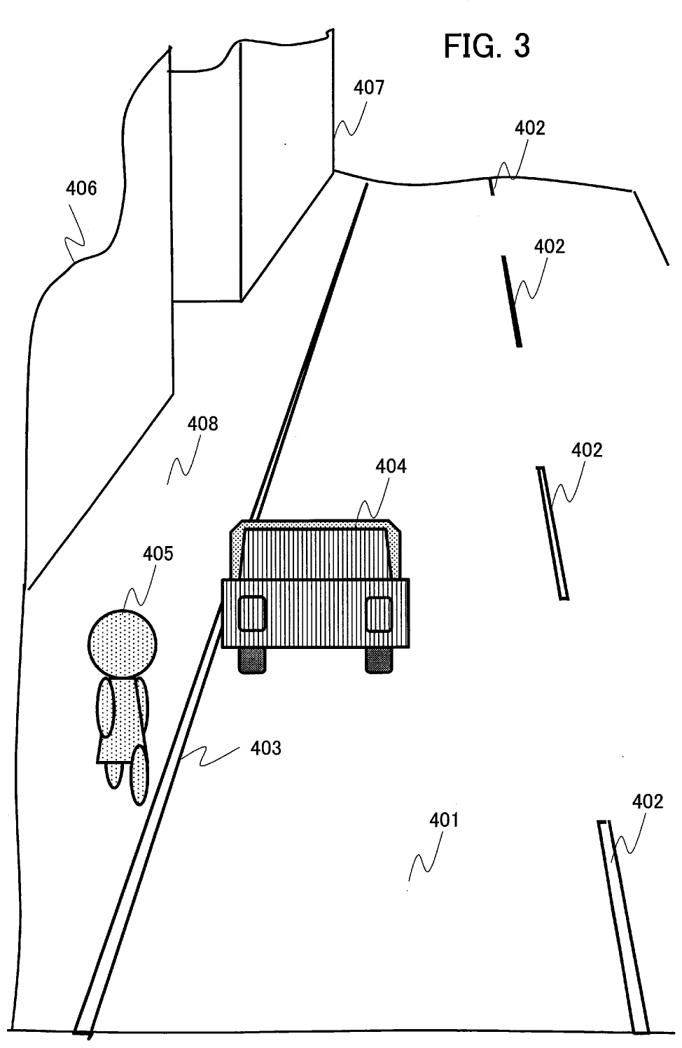

FIG. 3 is a diagram showing an example of data collected by the vicinal object detection device 110. The example of FIG. 3 is a simplified illustration of a still image acquired by the camera 112 by photographing the forward scene from the vehicle 100 at a certain time point. The still image of FIG. 3 includes a road 401, white lines 402 drawn on the road, a sidewalk step 403, a sidewalk 408, a leading vehicle 404, a pedestrian 405, and buildings 406 and 407. The still image may be transferred from the communication device 114 to the server 171 to perform a process of extracting objects captured in the still image, the server 171 may perform its own image recognition process, and the communication device 114 may receive the result of the recognition. Further, as another method, an image recognition process for extracting objects from the still image may be performed by an information processing device 181 of the operation aptitude judgment device 130. It is also possible to employ a method of judging objects such as the buildings 406 and 407 by matching with data of facilities existing in the vicinity by using position data acquired from the GPS 115 and the map data 117. Incidentally, the data acquired by the camera 112 is not limited to still image data but can also be motion video data.

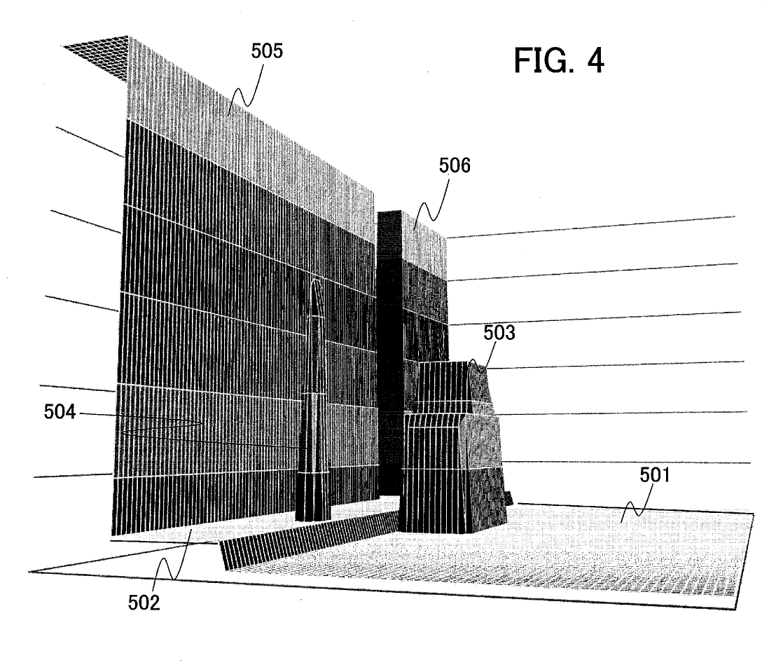

FIG. 4 is a diagram showing another example of data collected by the vicinal object detection device 110. The example of FIG. 4 schematically shows 3D data acquired by the radar 111, the 3D scanner 113 or the sensor 118 by detecting objects existing in the vicinity of the vehicle from the vehicle 100 at a certain time point. The 3D data in FIG. 4 expresses height data of objects existing in the vicinity by use of contour lines. The data in FIG. 4 is data acquired at the same time as the photographing of the still image in FIG. 3. The road 401 in FIG. 3 corresponds to a plane of data 501 in FIG. 3. Further, the sidewalk 408, the leading vehicle 404, the pedestrian 405 and the buildings 406 and 407 in FIG. 3 respectively correspond to data 502, 503, 504, 505 and 506 in FIG. 4. Since the white lines 402 in FIG. 3 are substantially at the same height as the road 401 and the sidewalk step 403 is substantially at the same height as the sidewalk 408, discrimination is not made as shown in FIG. 4 when the precision of detection is low. Once the height data of objects existing in the vicinity of the vehicle 100 is acquired as above, it is possible, based on the existence of the leading vehicle 404, the pedestrian 405 and the buildings 406 and 407 in FIG. 3 corresponding to the data 503, 504, 505 and 506 in FIG. 4, for example, to derive a range in which the situation behind these objects (situation in the part hidden behind) cannot be visually recognized, and such a range is determined as the perception difficulty space.

### (User Movement Detection Device 120)

The user movement detection device 120 shown in FIG. 2 is formed of various devices for collecting data necessary for detecting movement of the user in the vehicle 100. The user movement detection device 120 includes a user camera 121 and a user sensor 122, for example. The user camera 121 photographs the user and thereby acquires image data of the user in order to detect the user movement. Analyzing the image data of the user makes it possible to detect the user's body motion or the like. The user sensor 122 represents various types of sensors other than the camera for detecting the user movement. By using the user sensor 122, data that cannot be acquired by the user camera 121 can be acquired and more detailed and precise user movement can be detected. For example, by using a sight line detection sensor as the user sensor 122, the user's line of sight and the user's carefully watching direction can be detected. By providing a seat surface of a seat with a surface pressure sensor as the user sensor 122, the user's body motion or heartbeat can be detected. By using an infrared thermography as the user sensor 122, the user's surface temperature and its variations can be detected. Incidentally, the user movement detection device 120 may also be configured to include only one of the user camera 121 and the user sensor 122. Further, the user movement detection device 120 may include a plurality of user cameras 121 or a plurality of user sensors 122.

### (Operation Aptitude Judgment Device 130)

The operation aptitude judgment device 130 shown in FIG. 1 and FIG. 2 includes a storage device 182 and the information processing device 181 that makes the operation aptitude judgment on the user based on various measurement data obtained by the measurement by the vicinal object detection device 110 and the user movement detection device 120. The information processing device 181 makes the operation aptitude judgment on the user based on the measurement data. Specifically, the information processing device 181 includes a processor like a CPU (Central Processing Units), a GPGPU (General-Purpose computing on Graphics Processing Units) or an FPGA (Field-Programmable Gate Array). The storage device 182 includes a RAM (Random Access Memory) for temporarily storing data necessary for making the operation aptitude judgment on the user, a memory storing the operation aptitude judgment program to be executed by the information processing device 181, and so forth.

While a case where the information processing for making the operation aptitude judgment is performed in the operation aptitude judgment device 130 is described in the first embodiment for the simplicity of the description, it is unnecessary to perform all of the processing related to the operation aptitude judgment in the operation aptitude judgment device 130 as explained earlier in regard to the vicinal object detection device 110 and it is possible to employ a mode of distributed processing in which the processing is performed by the server 171 via the communication device 114 as needed. Thus, it is also possible to store the operation aptitude judgment program in the server 171.

### (Information Presentation Unit 140)

The information presentation unit 140 shown in FIG. 2 is a device used for presenting certain information to the user or a passenger. The information presentation unit 140 is a device presenting certain information by stimulating the senses of the human. A typical example of the information presentation unit 140 is a display device like a liquid crystal display for presenting image information. The information presentation unit 140 can include an HUD (Head-Up Display), a speaker for presenting audio information, a haptic display for stimulating the human's sense of touch by using various types of actuators, an olfactory display for stimulating the human's sense of smell by emitting smell, or the like.

### (Operation Unit 150)

The operation unit 150 shown in FIG. 2 is an operation device on which the user or a passenger performs operations for inputting user commands. The operation unit 150 is a device used for operating the vehicle 100 and various devices mounted on the vehicle 100. The operation unit 150 can include, for example, driving operation units used by the user for performing the operation of driving the vehicle and necessary for the driving control, such as a steering wheel, a brake pedal and an accelerator pedal. The driving operation units send out control commands to the vehicle control unit 160 which will be described later. Further, the operation unit 150 can include an information input operation unit such as a touch panel or a remote control. The information input operation unit is capable of sending out control commands to the information presentation unit 140 or various types of info/oration processing devices 181.

### (Vehicle Control Unit 160)

The vehicle control unit 160 shown in FIG. 2 is a control device for controlling the whole of the vehicle 100 in order to make the vehicle 100 operate. The vehicle control unit 160 controls the operation of the vehicle 100 based on the contents of operations performed by the user via the operation unit 150.

### (1-3) Operation

### (Algorithm)

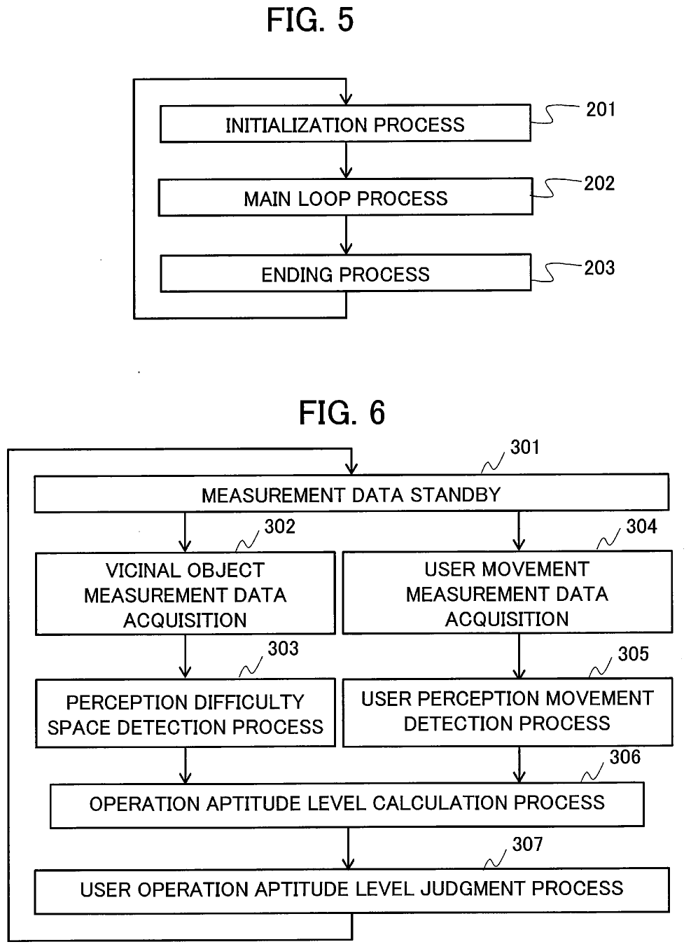

FIG. 5 shows a sequence indicating a basic process executed by the operation aptitude judgment device 130. When the vehicle 100 is started up, the operation aptitude judgment device 130 executes an initialization process 201. The initialization process 201 is a process required for appropriate operation of the operation aptitude judgment device 130.

When the initialization process 201 is completed, the operation aptitude judgment device 130 executes a main loop process 202. The main loop process 202 is an internal process repeated until the operation of the vehicle 100 ends.

When a process for ending the operation of the vehicle 100 starts, an interruption request for interrupting the main loop process 202 occurs, and the operation aptitude judgment device 130 receiving the interruption request as a trigger interrupts the main loop process 202 and executes an ending process 203. In the ending process, the operation aptitude judgment device 130 returns the operation aptitude judgment device 130 to an initializable state in preparation for the next startup of the vehicle 100.

### (Measurement Data Standby Process 301)

FIG. 6 is a sequence diagram showing details of an internal process of the main loop process 202 in the first embodiment. In the main loop process 202, a measurement data standby process 301 is executed first. In the measurement data standby process 301, the operation aptitude judgment device 130 requests the vicinal object detection device 110 and the user movement detection device 120 to provide their respective measurement data and stays on standby until the measurement data are provided. However, it is also possible to make the measurement data provision request only once at the first time and thereafter make the vicinal object detection device 110 and the user movement detection device 120 write the measurement data to predetermined regions in the storage device 182 by stream processing and thereby notify the operation aptitude judgment device 130 of the events.

### (User Perception Movement Detection Process 305)

When the user movement measurement data is provided from the user movement detection device 120, the operation aptitude judgment device 130 executes a user movement measurement data acquisition process 304 and thereby acquires the user movement measurement data. Thereafter, in a user perception movement detection process 305, the operation aptitude judgment device 130 detects what type of user perception movement the user is performing. In the first embodiment, a case of detecting a user perception movement by means of the sense of sight is described as an example. When a sight line detection sensor is installed as the user sensor 122 of the user movement detection device 120, the operation aptitude judgment device 130 in the user movement measurement data acquisition process 304 is capable of acquiring the user's viewpoint position, sight line direction, eye focal point position, etc. at the time point of measurement. Further, the operation aptitude judgment device 130 is capable of acquiring an image including the user's posture at the time point of measurement from the user camera 121 of the user movement detection device 120. The operation aptitude judgment device 130 is capable of executing the user perception movement detection process 305 by using these items of acquired data, thereby acquiring momentary conditions of the user perception movement such as the user's viewpoint position, sight line direction and focal point position, deriving the carefully watching direction and a visual field range from time series data of these momentary conditions, and deriving the user's attention and interest condition in a certain time window.

The detection result of the user perception movement detection process 305 may be stored in the storage device 182 to be referable in other process stages. Likewise, as to other processes, the processing result may be stored in the storage device 182 to be referable in other process stages.

In general, the user perception movement B detected in the user perception movement detection process 305 can be represented by a product set of a set {Dₚ₁, Dₚ₂, . . . , Dₚₗ} of data D_{p*} acquired in the user movement measurement data acquisition process 304 and a set {Bₚ₁, Bₚ₂, . . . , Bₚₘ} of detection results B_{p*} in the user perception movement detection process 305, that is, {Dₚ₁, Dₚ₂, . . . , Dₚₗ} ∩ {Bₚ₁, Bₚ₂, . . . , Bₚₘ}, where “l” and “m” are positive integers and “*” is a positive integer smaller than or equal to l or m. In the following description, to simplify the representation, D_{p*} is represented as B_{p*} for convenience and the user perception movement B is represented as B={Bₚ₁, Bₚ₂, . . . , Bₚₘ}.

When the measurement data is provided from the vicinal object detection device 110 in the measurement data standby process 301, a vicinal object measurement data acquisition process 302 is executed and the operation aptitude judgment device 130 acquires the measurement data. Thereafter, in a perception difficulty space detection process 303, the perception difficulty space that is difficult for the user to perceive is detected.

### (Basic Judgment Process Regarding Perception Difficulty Space)

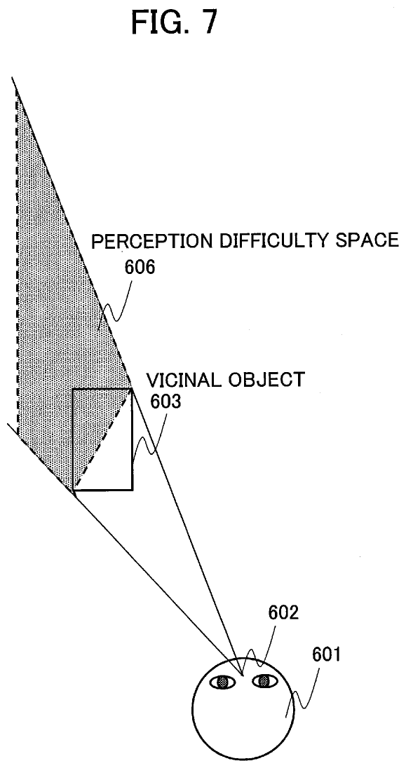

FIG. 7 is a diagram showing a concrete perception difficulty space detection process in regard to perception by means of the sense of sight. FIG. 7 shows a situation in which the viewpoint position 602 of the user 601 has been derived by executing the user perception movement detection process 305 in regard to the user 601 and a vicinal object 603 has been detected by the vicinal object detection device 110. In this case, with reference to the user's viewpoint position 602, it can be derived that a space beyond the outer circumference of the visible vicinal object 603 (a space hidden behind the vicinal object) is a perception difficulty space 606 caused by the vicinal object 603. FIG. 7 is expressed two-dimensionally in order to simplify the description. Even though the space in the real world is three-dimensional, the description of FIG. 7 is applicable also to three dimensions. Even in situations in which a plurality of vicinal objects exists, the perception difficulty space can be derived by performing a similar process for each vicinal object. Further, while the description of FIG. 7 is given in regard to the sense of sight, the present invention is not limited to the sense of sight or a single sensory organ. For example, the perception difficulty space may be obtained in regard to not the sense of sight but the sense of hearing, or obtained in regard to the sense of sight and the sense of hearing.

### (Importance Judgment Process Regarding Perception Difficulty Space)

In the perception difficulty space detection process 303 shown in FIG. 6, it is also possible to make a judgment on the importance of the detected perception difficulty space (dead space) in addition to the detection of the perception difficulty space.

As a scale of the importance, there exists “size of the perception difficulty space”. The size of the perception difficulty space can be regarded as an index indicating how much the perception difficulty space hides the perception object. In this case, the importance increases with the increase in the size of the perception difficulty space.

As another scale of the importance, there exists “distance between the perception difficulty space and the user or the vehicle”. This distance can be regarded as an index indicating grace for avoiding collision with a perception object when the perception object hiding in the perception difficulty space emerges, for example. In this case, the importance increases with the decrease in the distance.

As another scale of the importance, there exists “variation in the size of the perception difficulty space”. When the variation in the size is great, the variation can be regarded as an index of expansion of the range of the perception difficulty space with the passage of time. In this case, the importance increases with the increase in the variation in the size of the perception difficulty space.

As another scale of the importance, there exists “moving speed of the perception difficulty space”, “moving direction of the perception difficulty space” or “moving acceleration of the perception difficulty space”. The “moving speed”, the “moving direction” or the “moving acceleration” can be regarded as an index indicating grace for avoidance when a perception object hiding in the perception difficulty space emerges. In this case, when the movement is in a direction in which the perception difficulty space approaches, the importance increases with the increase in the moving speed and the increasing rate of the moving speed.

Further, as another scale of the importance, there exists a “level of difficulty of perception in the perception difficulty space”. This is because it is possible to find a hiding perception object with little labor when the level of difficulty of perception is low but the labor increases proportionally as the level of difficulty increases. For example, when a perception difficulty space is caused by obstruction of perception by roadside trees, it is possible to gain insight of the space behind the roadside trees through gaps between the roadside trees, and thus the level of difficulty is lower than that in cases of a perception difficulty space caused by a truck where it is totally impossible to gain insight of the space behind the truck. In this case, the importance increases with the increase in the difficulty of perception in the perception difficulty space.

Furthermore, when the remarkableness of the object as the factor causing the obstruction of perception in the perception difficulty space is lower than average, the probability that the user reflexively views the object as the factor is low, and thus the possibility that the user notices the perception difficulty space existing beyond the object (in a region behind the object as the factor) is also low. Thus, it can be interpreted that the importance of the perception difficulty space increases in such cases.

The level of the importance of the perception difficulty space may be either previously determined depending on the type of the object obstructing perception or dynamically calculated by use of values obtained by judging the presence/absence of a gap, permeability or remarkableness from the measurement data of the objects measured by the vicinal object detection device 110.

As above, the importance of the perception difficulty space is calculated by using a characteristic of the perception difficulty space itself and a characteristic derived from relationship between the perception difficulty space and another element such as the user or the vehicle. It is also possible to calculate the importance of the perception difficulty space not by using only one scale but by using a plurality of scales (a combination of two or more of the above-described scales of the importance) and values each obtained by multiplication by a weight coefficient.

(Importance Judgment Process Considering Relationship between Perception Difficulty Space and Operation)

Further, in the perception difficulty space judgment process and the importance judgment process, it is also possible to execute a judgment process in consideration of the contents of the operation the user should currently carry out. For example, the operation in the first embodiment is driving of a vehicle and it is necessary to recognize a vicinal object having a possibility of colliding with the traveling vehicle. In general, a vicinal object having a possibility of collision is an object stopped or moving on a plane at a height equivalent to the road on which the vehicle 100 is traveling, and thus an object existing at a certain height or higher has a low possibility of colliding with the vehicle and the possibility that the perception difficulty space caused by the object is hiding a general traffic object is low.

Similarly, also in regard to a perception difficulty space caused by an object at a position a certain distance or more apart from the position of the vehicle 100, or a space that is a certain distance or more apart from the position of the vehicle 100 in contrast with a perception difficulty space caused by an object existing within a certain distance, the possibility of collision is low since there is a sufficient grace distance for avoiding a potential object emerging from the space.

Further, even when a perception difficulty space exists within the aforementioned height or distance range, if an object blocking movement of objects exists between the perception difficulty space and the vehicle 100, the possibility that an object latent (hiding) in the perception difficulty space moves towards the vehicle is low. To show examples of specific situations, when a perception difficulty space is caused by a wall with no breaks, the possibility that a person or vehicle hidden behind the wall moves through the wall is low. Conversely, a gap through which a person can pass is generally formed in a line of vehicles continuously parked on the roadside. Since the line of vehicles has a break, there is a high possibility that an object hiding in the perception difficulty space caused by the line of vehicles moves towards the vehicle 100.

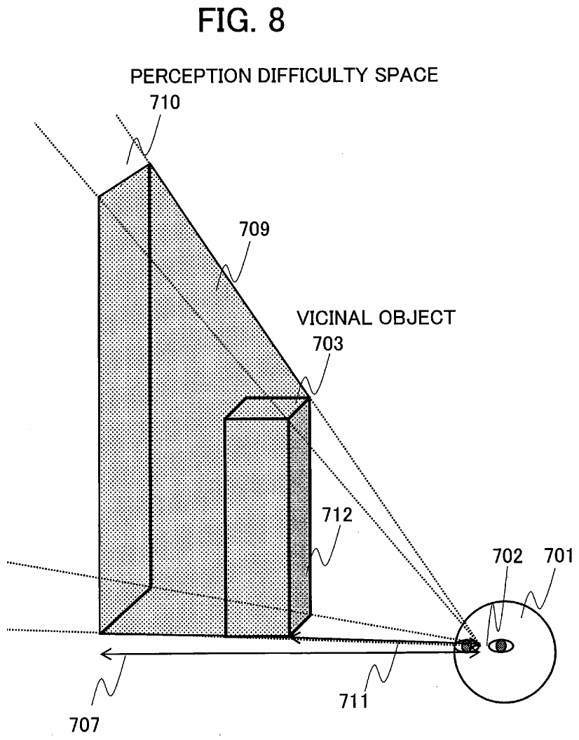

FIG. 8 is a diagram showing an example of a method of judging the importance of the perception difficulty space. In this example, a description will be given of a case where the user 701 is driving a vehicle with reference to a viewpoint position 702. A vicinal object 703 exists in the vicinity of the vehicle. A perception difficulty space 710 is caused by the vicinal object 703. A shortest distance 711 between the viewpoint position 702 and the vicinal object 703 is employed as a parameter that is used for calculating the importance of the perception difficulty space 710. The importance of the perception difficulty space 710 is inversely proportional to the shortest distance 711, or has a negative correlation with the shortest distance 711. Namely, as the user 701 approaches the vicinal object 703, the shortest distance 711 decreases and thus the importance of the perception difficulty space 710 increases.

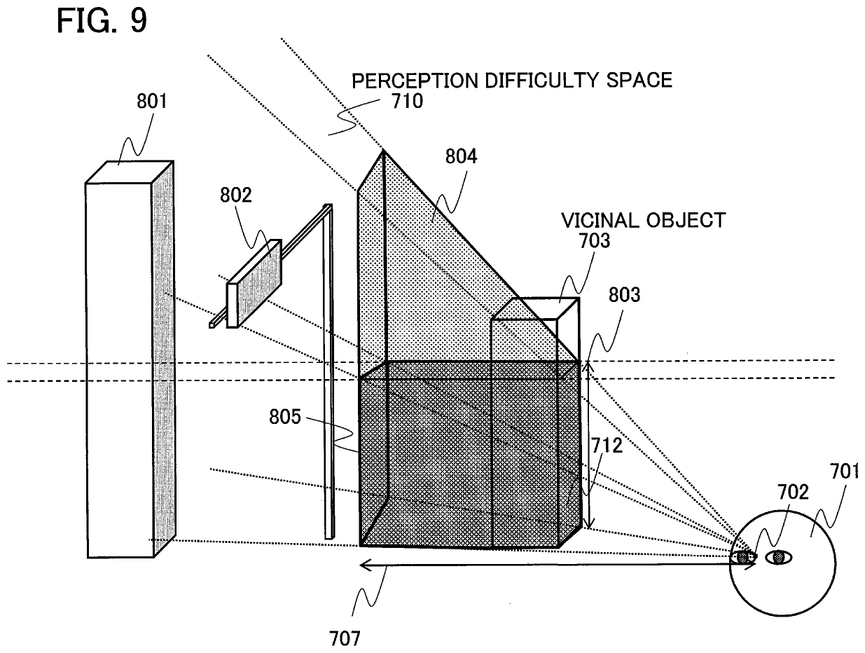

Further, as another parameter used for calculating the importance of the perception difficulty space 710, there exists the size of the perception difficulty space 710. The scale of the size of the perception difficulty space 710 is, for example, the area 712 of a surface of the perception difficulty space 710 on the side close to the user 701, or the volume of a part 709 of the perception difficulty space 710 included in a range from the surface of the perception difficulty space 710 on the side close to the user 701 to a surface that is a certain distance 707 apart from the user 701 (the volume of the hatched region in FIG. 9). When the importance of the perception difficulty space 710 is calculated by using these values, the importance is proportional to or has a positive correlation with the area 712 or the volume of the hatched region in FIG. 9.

FIG. 9 is a diagram showing another example of the method of judging the importance of the perception difficulty space 710. In FIG. 9, a vicinal object 801 and a signal 802 are added, in comparison to FIG. 8. FIG. 9 shows a case where a part of the perception difficulty space 710 existing above a height 803 (thinly hatched region) is assigned low importance and a part of the perception difficulty space 710 existing farther than a distance 707 (non-hatched region) is ignored in consideration of the contents of the operation of the user 701.

First, if the perception difficulty space is considered by taking the distance 707 into consideration, the vicinal object 801 and the signal 802 exist at positions farther than the distance 707, and thus the perception difficulty spaces caused by them are ignored. In contrast, the vicinal object 703 exists at a position closer than the distance 707, and thus it is judged that the perception difficulty space caused by the vicinal object 703 exists. Further, if the condition regarding the height 803 is considered, the perception difficulty space is divided into two types of spatial parts, namely, a spatial part (perception difficulty space) 805 existing in a range lower than or equal to the height 803 and a spatial part (perception difficulty space) 804 existing in a range higher than the height 803. In this case, the perception difficulty space 804 is judged to have a lower importance value than the perception difficulty space 805. When the user 701 advances and the signal 802 enters the range of the distance 707, a perception difficulty space is caused by the signal 802.

While the condition of setting the importance low for a perception difficulty space in a range higher than the height 803 and ignoring a perception difficulty space existing at a position farther than the distance 707 is set in the example of FIG. 9, the present invention is not limited to such a condition. The condition limiting the size of the perception difficulty space can be set as a different condition in consideration of the contents of the operation.

As above, even when the perception difficulty space exists, if the contents of the operation is taken into consideration, there are cases where it is appropriate to ignore the existence of a part of the perception difficulty space or to set the importance low for a part of the perception difficulty space by judging that there is no or almost no hindrance or danger to the operation. Conversely, there are also cases where it is appropriate to set the importance high for a part of the perception difficulty space when the operation is greatly hindered by the part of the perception difficulty space or there is a great risk of the hindrance.

Thus, in the process of the perception difficulty space judgment and the importance judgment with consideration for the contents of the operation, the contents of the operation is not taken into consideration at first; after the perception difficulty space is detected, filtering the detected perception difficulty space or setting the importance to the detected perception difficulty space is performed according to whether or not a condition specified based on the contents of the operation is satisfied, and thus the process of the perception difficulty space judgment and the importance judgment with consideration for the contents of the operation can be achieved. The condition specified based on the contents of the operation in this case is not limited to the height from the road surface, the distance from the vehicle, or the presence/absence of an object blocking the emergence of an object from the perception difficulty space; it is also possible to use different conditions based on the contents of the operation.

The importance of the perception difficulty space detected by the perception difficulty space detection process 303 (FIG. 6) described above can be summarized as follows:

In regard to a certain perception difficulty space X, when weights based on characteristics g_{Xi} of the perception difficulty space X itself, such as the shape and size of the perception difficulty space X itself, the distance between the perception difficulty space X and the vehicle driven by the user and variations as their time series variations, are represented as w(g_{Xi}) (i: positive integer),

weights based on perceptual characteristics p_{Xi} of an object as the factor causing the perception obstruction, such as the permeability or a gap ratio as an influence of the object as the factor causing the perception obstruction and the remarkableness of the object as the factor causing the perception obstruction, are represented as w(p_{Xi}), and

weights based on conditions c_{Xi} considering the contents of the operation carried out by the user are represented as w(c_{Xi}),

the importance Wx of the perception difficulty space X is represented by the following expression:

W_{X}=ΣᵢW(g_{Xi})+ΣᵢW(p_{Xi})+ΣᵢW(c_{Xi}) expression 1

Further, the perception difficulty space X in this case can be represented by a set of its own characteristics as:

G_{X}={g_{X1},g_{X2}, . . . ,g_{Xn}}.

While the importance Wx in this example is represented by the total value on the assumption that the weights w(g_{Xi}), w(p_{Xi}) and w(c_{Xi}) are independent of each other, the calculation of the importance Wx is not limited to the expression 1. The importance Wx may also be calculated by using the above-described characteristics or the like. For example, it is described earlier that the perception difficulty space is dismissed when a condition c_{*i} considering the contents of the operation carried out by the user satisfies a certain condition. In that case, assuming that a threshold value regarding the condition c_{*i} is TC_{*i}, for example, the importance Wx can be represented by the following expressions 2 and 3, for example:

W_{X}=0(∃c_{Xi}:c_{Xi}<TC_{*i}) expression 2

W_{X}=ΣᵢW(g_{Xi})+ΣᵢW(p_{Xi})+ΣᵢW(c_{Xi})

(∀c_{Xi}:c_{Xi}≥TC_{*i}) expression 3

### (Operation Aptitude Level Calculation Process 306)

In an operation aptitude level calculation process 306 in FIG. 6, the operation aptitude level indicating how appropriately the user at that time point can carry out the operation is calculated based on the perception difficulty space and its importance detected by the perception difficulty space detection process 303 and the user perception movement detected by the user perception movement detection process 305.

In the first embodiment, whether the user has anticipated a perception object hiding in a visual perception difficulty space or not is used as an example of the scale of the operation aptitude.

### (Example of Basic Operation Aptitude Level Calculation Process)

When the aforementioned anticipation of a perception object has occurred appropriately, a correlation occurs between the perception difficulty space and the user perception movement. Specifically, there are cases where the perception difficulty space and the user's sight line vector intersect with each other, a movement vector of the perception difficulty space and the user's sight line movement vector are similar to each other, the user's sight line vector changes to intersect with the perception difficulty space when a sharp change occurred in one or more characteristics of the perception difficulty space, and so forth.

Incidentally, it is also possible to use a different method such as deriving a correlation with the perception difficulty space based on the number of times or the frequency of sight line movement or the increase or decrease in a sight line retention time.

Such a correlation can be derived arithmetically by acquiring the data under consideration as time series data and using correlation coefficients or the like. The correlation CR_{X} with a certain perception difficulty space X can be represented as the following expression 4 by using a characteristic G_{X} of the perception difficulty space X and the user perception movement B:

CR_{X}=Σᵢαᵢƒᵢ(G_{X},B) expression 4

where “fᵢ( )” is a function for calculating a value representing a relationship such as the aforementioned correlation between the perception difficulty space X and the user perception movement B according to a certain criterion i, and αᵢ is a weight in regard to the criterion i. Further, the user perception movement B is not limited to a user perception movement at a certain particular time point; the user perception movement B may be described as time series data within a certain time series window. The same applies to the characteristic G_{X} of the perception difficulty space X.

The magnitude of the value of CR_{X} can be regarded as a scale indicating how much the user is conscious of the perception difficulty space X to perceive the perception difficulty space X. For example, it can be interpreted that the operation aptitude level judged based on the perception difficulty space X is high if the value is large and the operation aptitude level is low if the value is small. The average value of the correlations CR_{X} regarding all perception difficulty spaces at that time point is represented by the following expression 5:

CR=ΣᵢCR_{X}/N expression 5

This is a scale indicating whether the user is trying to exhaustively perceive the perception difficulty spaces at that time point. Here, N represents the number of perception difficulty spaces detected at that time point (positive integer).

### (Example of Operation Aptitude Level Calculation Process Using Importance of Perception Difficulty Space)

It is also possible to obtain CR_{X} by considering the importance of each perception difficulty space calculated from the perception difficulty space, which can be formulated as the following expression 6:

CR_{X}=ΣᵢαᵢW_{X}ƒᵢ(G,B) expression 6

The operation aptitude level calculation process 306 calculates CR_{X} or CR explained above as one of the operation aptitude levels. By using at least the calculation result, a user operation aptitude level judgment process 307 for judging the operation aptitude level of the user is executed and the user's operation aptitude at that time point is judged. After completion of the user operation aptitude level judgment process 307, the process returns to the measurement data standby process 301 and repeats the processing. When an ending process of the vehicle 100 starts, an interruption process is executed immediately irrespective of which process in FIG. 6 is in progress, by which the main loop process 202 can be interrupted.

### (Cases Including Counter-Obstruction Perception Movement)

The methods described so far are not limited to a certain normal user perception movement. As mentioned earlier, user perception movements include counter-obstruction perception movements of actively trying to perceive the perception difficulty space, and operation aptitude level calculation considering a characteristic of the counter-obstruction perception movement is also possible. In that case, not only data regarding the sense of sight but also data regarding the body motion are acquired as the data acquired by the user movement measurement data acquisition process 304, and it is judged in the user perception movement detection process 305 whether or not the user is performing the counter-obstruction perception movement and if the user is performing the counter-obstruction perception movement, the level of the counter-obstruction perception movement is also judged based on a correlation between the increase or decrease in the body motion and the normal user perception movement by using the data regarding the body motion. The level BC of the counter-obstruction perception movement related to the body motion is detected in the user perception movement detection process 305, the level BC is paired with the user perception movement B detected at the same time, and the data to which the level BC of the counter-obstruction perception movement has been added is handed over to the operation aptitude level calculation process 306.

Further, in regard to changes in reaction sensitivity to a sensory organ, the degree of the decrease in the perceptual sensitivity of the sensory organ can be calculated based on a perception difficulty space other than a perception difficulty space to which the user is currently directing attention or another vicinal environment, a reaction time of each sensory organ to their changes, and so forth. The level SC of the counter-obstruction perception movement accompanied by a change in the reaction sensitivity to a sensory organ is detected in the user perception movement detection process 305, paired with the user perception movement B detected as well or the level BC of the counter-obstruction perception movement accompanied by a body motion change, and is handed over to the operation aptitude level calculation process 306.

The method of calculating the operation aptitude level in the operation aptitude level calculation process 306 by using SC and BC will be explained below. SC and BC are paired with the user perception movement B at that time, and it is possible to judge to which perception difficulty space X the counter-obstruction perception movement is directed based on the user perception movement B. For example, the judgment is made based on the sight line vector in cases of the sense of sight, based on the frequency range in cases of the sense of hearing, and so forth. The object of the counter-obstruction perception movement can be, in more generic representation, represented as stochastic representation, namely, a probability value CP_{X} of a case where the perception difficulty space X is the object of the counter-obstruction perception movement.

The correlation CR_{X} with a certain perception difficulty space X can be represented by the following expression 7:

CR_{X}=CW(B,SC,BC,G_{X})·CP_{X}Σᵢαᵢƒᵢ(G_{X},B)+CC(B,SC,BC,G_{X}) expression 7

where CW(B, SC, BC, G_{X}) and CC(B, SC, BC, G_{X}) respectively represent the weight and the intercept when the level of the counter-obstruction perception movement exerts an influence on the correlation Σᵢαᵢfᵢ(G_{X}, B). Specifically, the weight or the intercept takes on a large value if the counter-obstruction perception movement is directed towards the perception difficulty space X, or conversely takes on a small value or a negative value depending on the situation if the counter-obstruction perception movement is not directed towards the perception difficulty space X. It is unnecessary to employ both of the weight and the intercept at the same time; it is possible to employ one of the weight and the intercept or neither of the weight and the intercept. The weight and the intercept may be determined based on a certain predetermined table, or calculated each time in a model-based method by constructing a certain model. Further, the counter-obstruction perception movement does not necessarily have to be considered constantly; it is also possible to reduce the processing load by calculating the correlation CR_{X} in consideration of the counter-obstruction perception movement only when there exists at least one perception difficulty space.

### (Cases Where Operation is Considered)

Still another method of the operation aptitude level calculation will be described below. Depending on the contents of the operation that the user should carry out, there can be cases where consciousness of perception in regard to the perception difficulty space is biased. In the driving of a vehicle in the first embodiment, considering the fact that the user should pay attention to an object rushing out onto the road from a dead space, the user does not need to thoroughly perceive the perception difficulty space, and as far as the perception difficulty space is concerned, the perception should be biased and concentrated on the vicinity of the boundary of the perception difficulty space.

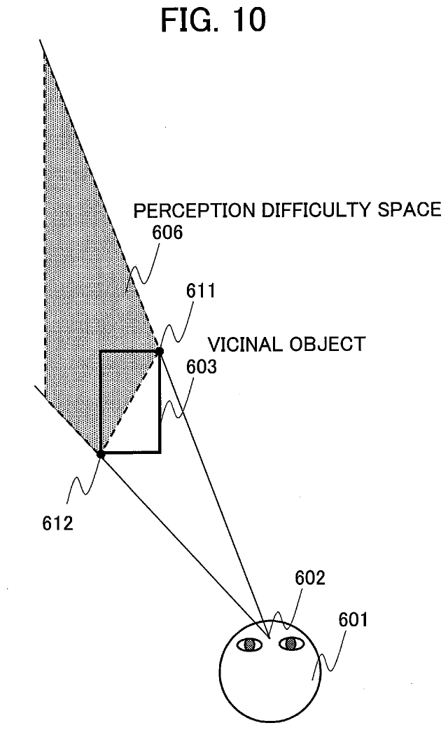

The method of the operation aptitude level calculation considering the contents of the operation will be described below with reference to FIG. 10. FIG. 10 is a diagram showing a situation in which there exists a perception difficulty space 606 caused by a vicinal object 603 in regard to the viewpoint position 602 of the user 601 as a reference point. In this case, passage of an object through the vicinal object 603 it is generally difficult, and thus there is a low possibility that a person or the like as a perception object passes through the vicinal object 603 and emerges from a plane surface including a line segment connecting points 611 and 612. In contrast, there is a high possibility that a person or the like as a perception object emerges through the vicinity of the point 611 or the point 612. To sum up, the level to which the user should be conscious of the perception regarding the perception difficulty space (perception importance level) is not uniform and a bias can occur depending on the contents of the operation.

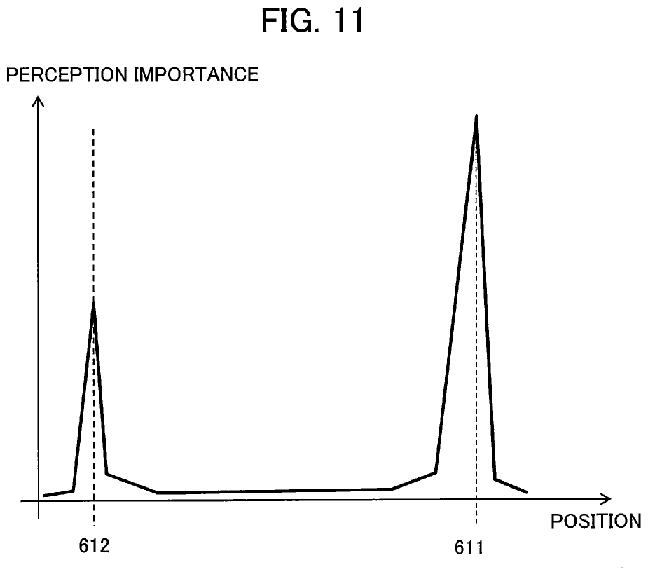

FIG. 11 is a diagram showing an example of the perception importance level in regard to each position on the plane surface including the line segment extending from the point 612 to the point 611 on the vicinal object 603. In this example, the importance level is the highest in the vicinity of the point 611 and the second highest in the vicinity of the point 612. In this case, the operation aptitude level calculation considering the contents of the operation becomes possible by calculating the operation aptitude level to be higher in cases where the line of sight is directed towards the vicinity of the point 611 or the point 612 than in cases where the line of sight is directed towards a point between the point 611 and the point 612. This can be regarded as one of the criteria i in the calculation of the correlation CR_{X}.

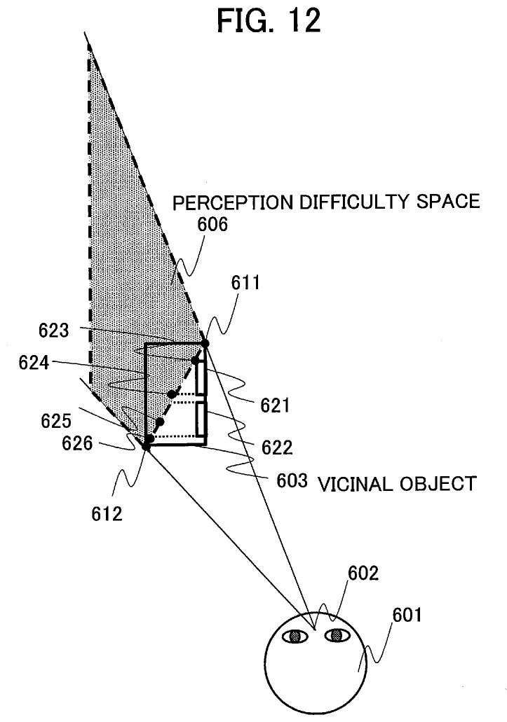

Another method of the operation aptitude level calculation considering the contents of the operation will be described below with reference to FIG. 12. FIG. 12 is a diagram showing a situation in which the vicinal object 603 in FIG. 10 is another vehicle and there exists a perception difficulty space 606 caused by the vicinal object 603 in regard to the viewpoint position 602 of the user 601 as the reference point. Information for judging another vehicle as an attribute of the vicinal object 603 can be implemented by performing data clustering on the data acquired from the vicinal object detection device 110 by using algorithm such as machine learning. The other vehicle 603 has doors 621 and 622 on its side face and there is a possibility that a passenger comes out from the inside of the other vehicle 603. Thus, differently from the situation shown in FIG. 10, the user should direct the line of sight not only towards the vicinity of the points 611 and 612 but also towards the vicinity of line segments connecting points 623 and 624 and points 625 and 626 obtained by projecting the doors 621 and 622 onto the line segment connecting the points 611 and 612.

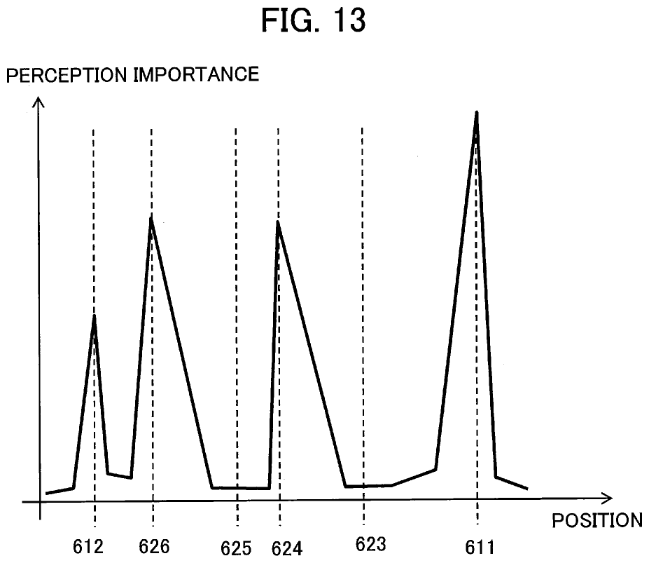

FIG. 13 is a diagram showing an example of the perception importance level in regard to each position on the plane surface including the line segment extending from the point 612 to the point 611 on the vicinal object 603 in the situation of FIG. 12. In this example, the perception importance level is high on the line segments connecting the points 623 and 624 and the points 625 and 626 corresponding to the doors 621 and 622. In this example, the perception importance level monotonically decreases from the point 626, 624 on the side close to the user 601 towards the point 625, 623 on the side far from the user 601 on both line segments. This is because the other vehicle 603 is parked facing the direction the user 601 faces and each door 621, 622 opens on the side close to the user and accordingly the perception importance level is high at the corresponding point 626, 624.

### (1-4) Effect

As described above, in the operation aptitude judgment device 130, the operation aptitude judgment method and the operation aptitude judgment program according to the first embodiment, it becomes possible to judge whether the user at that time point is in a condition suitable for carrying out the operation or not based on the relationship between how much the user is conscious of the perception difficulty space, as a space in which perception necessary for carrying out the operation is obstructed, and the user's perception action. In this case, since the perception difficulty space itself cannot be perceived, it is easy to distinguish between reflexive reaction due to remarkableness of the perception difficulty space itself and reaction as a result of recognition for carrying out the operation such as risk anticipation in regard to perception difficulty. Accordingly, the operation aptitude level indicating in how suitable condition the user is to perform the operation can be judged precisely without imposing a burden on the user.

### (2) Second Embodiment

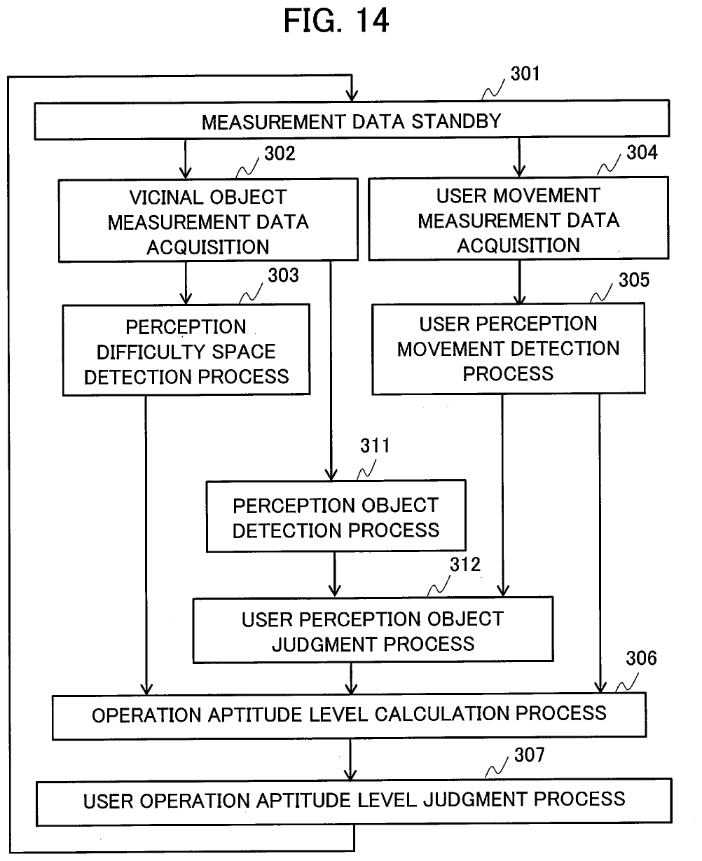

FIG. 14 is a sequence diagram showing details of another internal process of the main loop process 202 in FIG. 5. In FIG. 14, each process identical with a process in FIG. 6 is assigned the same reference character as in FIG. 6. In the second embodiment, the description will be given mainly of features different from those in the first embodiment. The internal process shown in FIG. 14 differs from the internal process shown in FIG. 6 (first embodiment) in that a perception object detection process 311 and a user perception object judgment process 312 are added. Further, the operation aptitude judgment device in the second embodiment differs from that in the first embodiment in including a perception object detection unit 134 (FIG. 1) that executes the perception object detection process 311 and a user perception object judgment processing unit 135 (FIG. 1) that executes the user perception object judgment process 312. By adding these processes, in the operation aptitude judgment device, the operation aptitude judgment method and the operation aptitude judgment program according to the second embodiment, perception objects existing in the vicinity of the user are detected, a judgment is made to determine which one of the detected perception objects has been perceived by the user, and the operation aptitude judgment on the user is made by using information on the perceived object (result of the judgment). Except these features, the first embodiment is the same as the second embodiment. Incidentally, FIG. 1 and FIG. 2 are also referred to in the description of the second embodiment.

In the perception object detection process 311 shown in FIG. 14, an object that the user should perceive when the user performs the operation is detected based on the information regarding the vicinal objects acquired in the vicinal object measurement data acquisition process 302.

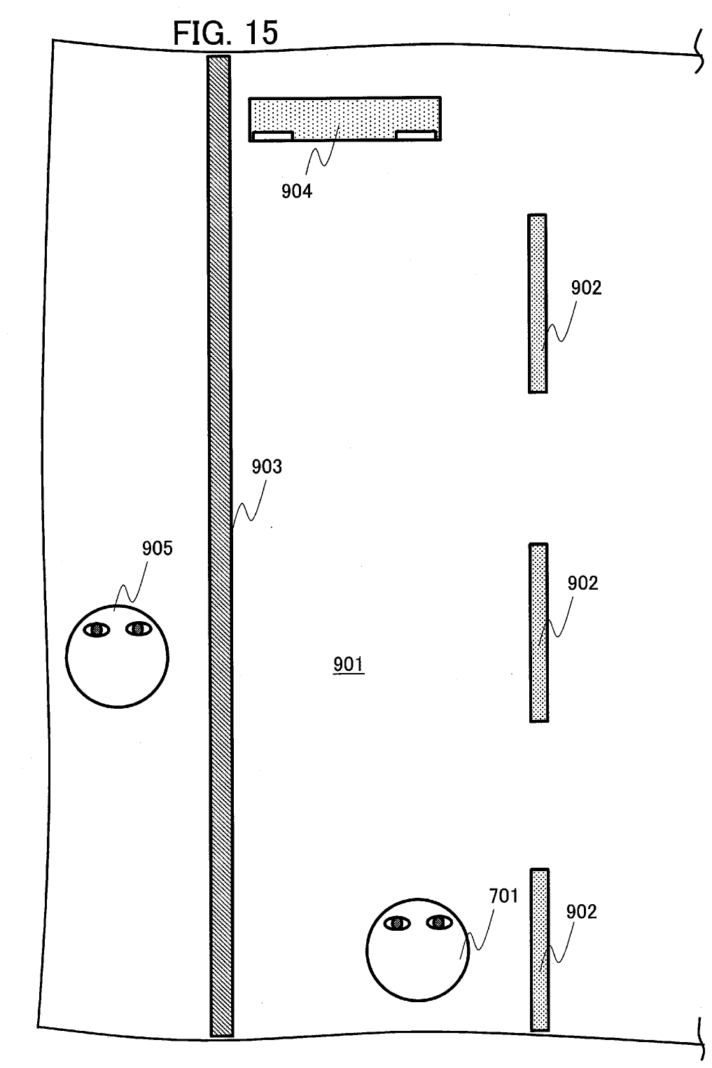

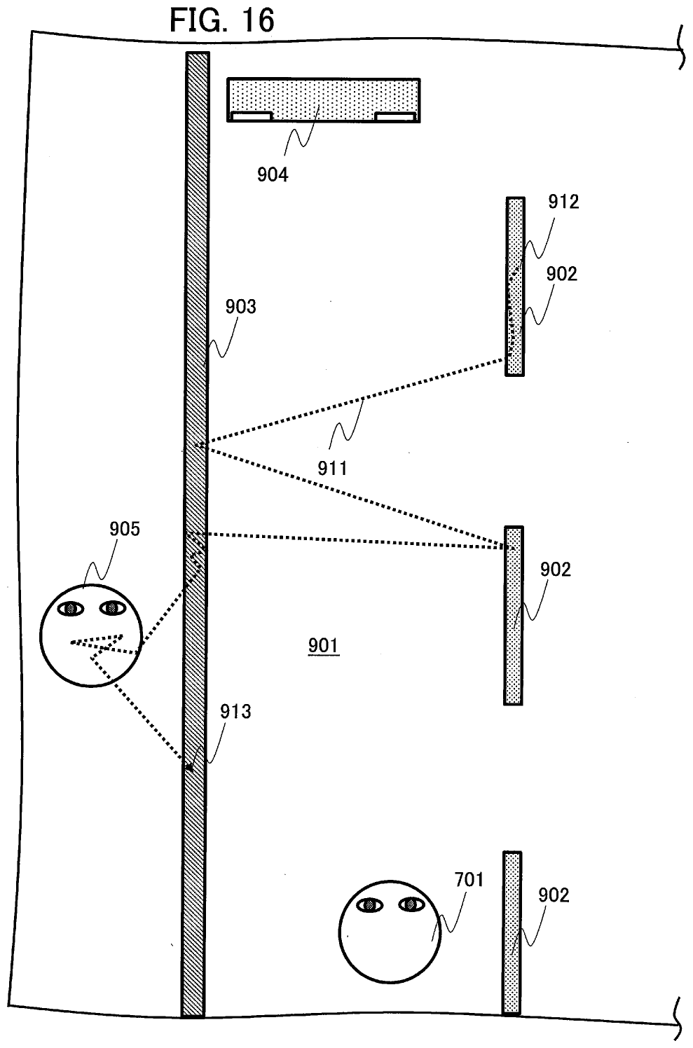

FIG. 15 is a diagram for explaining the perception object detection process 311 in FIG. 14. As objects in the vicinity of the user 701, there exist a road 901, white lines 902, a sidewalk step 903, a vehicle 904 traveling in front, and a pedestrian 905 walking on the sidewalk, and further exist various vicinal objects such as the sky, a cloud, a bird and an airplane. The vicinal object detection device 110 acquires data of these objects in the vicinity as a series of data without distinction.

Normally, when the user 701 carries out an operation, it is not necessary for the user 701 to recognize all of the vicinal objects; it is permissible if the user 701 recognizes part of a lot of vicinal objects. The vicinal objects that the driver as the user 701 should recognize are, for example, the white lines 902, the sidewalk step 903, the vehicle 904 traveling in front, and the pedestrian 905. Thus, in the perception object detection process 311 in FIG. 14, information on vicinal objects that the user does not need to recognize, such as the road 901, is removed by performing filtering. The filtering can be carried out by using object recognition technology based on a known algorithm such as machine learning for detection data of vicinal objects acquired from the vicinal object detection device 110. As the result of the filtering, attribute information on objects that should be recognized (perceived) at the time point of the filtering, such as the type, shape, position and size of each object, can be extracted. It is also possible to extract variations in the attribute information by acquiring the attribute information on the detected objects in a time series and making comparison between pieces of the attribute information that differ in the detection time.

In the user perception object judgment process 312 in FIG. 14, the probability that the objects that should be perceived have already been perceived by the user is judged based on a list of the attribute information on the objects that should be perceived as the detection result of the perception object detection process 311 and the information on the user perception movement detected in the user perception movement detection process 305.

FIG. 16 is a diagram for explaining the user perception object judgment process 312 in FIG. 14. FIG. 16 is a diagram in which movement time series data 911 of a position at the end of the line of sight (position on the object) detected in the perception object detection process 311 is superimposed on FIG. 15. In the movement time series data 911 of the position at the end of the line of sight, a point 912 is the starting point, a changing point of a line segment (a spot where the line segment is bent) indicates the position at the end of the line of sight detected next, and a point 913 is the latest position at the end of the line of sight. In this case, it is indicated that the visual attention has moved from a white line 902 successively to the sidewalk step 903, a white line 902, the sidewalk step 903, the pedestrian 905 and the sidewalk step 903. In such a case, it can be interpreted that the white lines 902, the sidewalk step 903 and the pedestrian 905 have already been recognized by the user whereas the vehicle 904 traveling in front has not been recognized by the user, for example.

As the level of the recognition, it is possible to use a retention time of the line of sight, an elapsed time since the line of sight moved away, or weighting coefficients considering both of these times. Specifically, there are parameters related to the user's perception action, such as the number of times the line of sight is directed towards a certain perception object Y, a retention time for which the line of sight is directed towards the perception object Y, an elapsed time after the line of sight is shifted away from the perception object Y, or a combination of some of these, and when the parameter is represented as zi and the weight of the parameter zi is represented as W(zi), a scale P(Y) indicating whether the user has recognized the perception object Y can be represented by the following expression 8:

P(Y)=ΣᵢW(zᵢ) expression 8

The following is an example of calculating the scale indicating whether the user has recognized each perception object in FIG. 16 by using the number of times the user's line of sight was directed towards the perception object as the parameter related to the user's perception object:
P(white line 902)=5
P(sidewalk step 903)=6
P(vehicle 904 traveling in front)=0
P(pedestrian 905)=4
   In this case, the sidewalk step 903 is judged to be the object of the highest level of perception (i.e., the highest scale of recognition).

When the maximum value of the number of times the line of sight is directed consecutively is used as another parameter, for example, an example of calculating the scale indicating whether the user has recognized each perception object in FIG. 16 is as follows:
P(white line 902)=4
P(sidewalk step 903)=4
P(vehicle 904 traveling in front)=0
P(pedestrian 905)=4
   In this case, the perception objects other than the vehicle 904 traveling in front are judged to be at the same level of perception (i.e., at the same level in the scale of recognition).

The parameters regarding the user's perception action are not limited to the above-described parameters; it is also possible to define other parameters.

In the second embodiment, the operation aptitude level calculation process 306 is executed by using the outputs of the user perception object judgment process 312, the perception difficulty space detection process 303 and the user perception movement detection process 305. In the example described in the first embodiment, the correlations CR_{X} between the perception difficulty spaces X and the user perception movement are obtained by use of the perception difficulty space detection process 303 and the user perception movement detection process 305 and the operation aptitude level is calculated from these correlations CR_{X}. In contrast, in the second embodiment, an index for obtaining the operation aptitude level is calculated by further using the output of the user perception object judgment process 312. In the user perception object judgment process 312, in regard to each vicinal object, a value based on the scale indicating how much the user has recognized the vicinal object is outputted.

For example, when the scale regarding an object U is represented as P(U), the total value V=Σ_{U}P(U) of P(U) can be interpreted as a value indicating how much the user has recognized all objects existing in the vicinity at that time point. This total value V is an example of the operation aptitude level. This calculation method is just an example and a different calculation method may be employed. For example, it is also possible to assign a weight to each scale P(U) according to the type of the object U or a characteristic of the object U other than the type and obtain a weighted sum total value as the operation aptitude level.

Further, when an object U exists in the vicinity of (close to) a certain perception difficulty space, there are cases where a part of the object U is hidden by the perception difficulty space. There are also cases where another object Y that does not exist until immediately before emerges from the vicinity of a certain perception difficulty space. As above, objects distributed in the vicinity of a perception difficulty space can be interpreted as perception objects having priority over other objects, and it is possible in such cases to increase the weighting of the scale P(U) and obtain a weighted sum total value as the operation aptitude level.

As described above, in the operation aptitude judgment device, the operation aptitude judgment method and the operation aptitude judgment program according to the second embodiment, the operation aptitude level indicating in how suitable condition the user is to perform an operation can be judged still more precisely without imposing a burden on the user.

### (3) Modifications

While cases where the user is the driver of an automobile have been described in the above first and second embodiments, the vehicle driven by the user can be a vehicle other than an automobile. The vehicle can be, for example, a mobile object such as a bicycle, a motorcycle or a trolley. The operation to which the present invention is applicable is not limited to the operation of a mobile object and can be an operation other than the operation of a mobile object such as the operation of a facility or a machine. For example, when the operation that the user should perform is a machining operation using a machine tool, it is possible to regard shavings as perception objects, regard a region scattered with fine shavings as a perception difficulty space, and assign the material or size of the shaving as a parameter of the importance of the perception difficulty space. In this case, the user's visually checking the machine tool or its vicinity before touching in order to counter low visibility due to the fineness of the shavings can be regarded as the counter-obstruction perception movement, for example, and the number of times of the movement, the frequency of the movement, the retention time of the movement, a combination of some of these, or the like can be regarded as the level of the counter-obstruction perception movement.

Further, while examples of using perception objects perceived by the sense of sight and perception difficulty spaces in which perception by the sense of sight is difficult have been described in the above first and second embodiments, the perception used in the present invention is not limited to the sense of sight; the present invention is applicable also to other senses such as the sense of hearing, the sense of touch and the sense of taste. For example, when the operation that the user should perform is a machining operation using a machine tool, it is possible to regard abnormal sound of the machine operated by the user as a perception object, regard other sounds such as operation sound when the machine is operating normally and sound emitted from a machine operated by another operator as perception difficulty spaces, and define the importance of each perception difficulty space as the degree of similarity to the abnormal sound of the machine, the sound level, the direction of the source of the sound, a combination of some of these, or the like. In this case, in correlation with the importance of the perception difficulty space, the user's stopping an operational movement, visually checking the machine tool and its vicinity, or the like can be regarded as the counter-obstruction perception movements, for example, and the number of times of the movement, the frequency of the movement, the retention time of the movement, or the like can be regarded as the level of the counter-obstruction perception movement.

### DESCRIPTION OF REFERENCE CHARACTERS

100: vehicle, 110: vicinal object detection device, 120: user movement detection device, 130: operation aptitude judgment device, 131: user perception movement detection unit, 132: perception difficulty space detection unit, 133: operation aptitude level calculation unit, 134: perception object detection unit, 140: information presentation unit, 181: info/notion processing device, 182: storage device, 601, 701: user, 603, 703: vicinal object.

1-15. (canceled)

16. An operation aptitude judgment device that judges an operation aptitude level indicating in how suitable condition a user is to perform a planned operation that should be carried out, the operation aptitude judgment device comprising:
a perception difficulty space detection unit to detect perception difficulty space in which a perception object as an object that the user should perceive when the user performs the planned operation is difficult for the user to perceive based on vicinal object information acquired from a vicinal object detection device that detects a vicinal object existing in a vicinity of the user, and importance of the perception difficulty space;
a user perception movement detection unit to detect a user perception movement, as a movement of the user when the user tries to perceive the perception object, a counter-obstruction perception movement as a user perception movement when the user tries to perceive a potential perception object as a perception object that can exist in the perception difficulty space, and a level of the counter-obstruction perception movement, based on user movement information acquired from a user movement detection device that detects a movement of the user; and
an operation aptitude level calculation unit to calculate the operation aptitude level of the user based on the perception difficulty space detected by the perception difficulty space detection unit, the importance of the perception difficulty space, the user perception movement detected by the user perception movement detection unit, the counter-obstruction perception movement, and the level of the counter-obstruction perception movement.

17. The operation aptitude judgment device according to claim 16, further comprising a perception object detection unit to detect the perception object,
wherein the operation aptitude level calculation unit calculates the operation aptitude level of the user based on the perception object detected by the perception object detection unit, the perception difficulty space detected by the perception difficulty space detection unit, the importance of the perception difficulty space, the user perception movement detected by the user perception movement detection unit, the counter-obstruction perception movement, and the level of the counter-obstruction perception movement.

18. The operation aptitude judgment device according to claim 16, wherein the perception difficulty space detection unit acquires information indicating contents of the planned operation and determines the perception difficulty space based on the contents of the planned operation.

19. The operation aptitude judgment device according to claim 17, wherein the perception object detection unit acquires information indicating contents of the planned operation and determines the perception object based on the contents of the planned operation.

20. The operation aptitude judgment device according to claim 16, wherein the perception difficulty space detection unit judges a dead space that is not directly visible from a position of the user as the perception difficulty space.

21. The operation aptitude judgment device according to claim 17, wherein the perception object detection unit judges an object existing in a space that is not directly visible from a position of the user as the perception object.

22. The operation aptitude judgment device according to claim 16, wherein the user perception movement detection unit detects a movement of the user's line of sight as the user perception movement.

23. The operation aptitude judgment device according to claim 16, wherein the user perception movement detection unit changes the operation aptitude level based on one or more of a number of times of user sight line movement in which the user's line of sight is directed towards a region including the perception difficulty space and a vicinity of the perception difficulty space, a frequency of the user sight line movement, and a retention time for which the user's line of sight is directed towards the region.

24. The operation aptitude judgment device according to claim 16, wherein
the user perception movement detection unit detects a counter-obstruction perception movement as a user perception movement when the user tries to perceive a potential perception object as a perception object that can exist in the perception difficulty space and a level of the counter-obstruction perception movement, and
the operation aptitude level calculation unit changes the operation aptitude level based on at least one of the counter-obstruction perception movement and the level of the counter-obstruction perception movement.

25. The operation aptitude judgment device according to claim 16, wherein
the perception difficulty space detection unit
judges a dead space that is not directly visible from a position of the user as the perception difficulty space, and
determines the importance of the perception difficulty space based on one or more of size of the dead space, a position of the dead space, distance from the user to the dead space, moving speed of the dead space, and moving acceleration of the dead space.

26. The operation aptitude judgment device according to claim 23, wherein the level of the counter-obstruction perception movement is changed based on at least one of a number of times of user sight line movement in which the user's line of sight is directed towards a vicinity of the perception difficulty space on a side close to the user, a frequency of the user sight line movement, and a retention time for which the user's line of sight is directed towards the vicinity.

27. The operation aptitude judgment device according to claim 16, wherein the perception difficulty space detection unit detects the perception difficulty space that is detected within a predetermined range from the user and does not detect the perception difficulty space outside the predetermined range.

28. An operation aptitude judgment method of judging an operation aptitude level indicating in how suitable condition a user is to perform a planned operation that should be carried out, the operation aptitude judgment method comprising:
detecting a perception difficulty space in which a perception object as an object that the user should perceive when the user performs the planned operation is difficult for the user to perceive based on vicinal object information acquired from a vicinal object detection device that detects a vicinal object existing in a vicinity of the user, and importance of the perception difficulty space;
detecting a user perception movement, as a movement of the user when the user tries to perceive the perception object, a counter-obstruction perception movement as a user perception movement when the user tries to perceive a potential perception object as a perception object that can exist in the perception difficulty space, and a level of the counter-obstruction perception movement, based on user movement information acquired from a user movement detection device that detects a movement of the user; and
calculating the operation aptitude level of the user based on the detected perception difficulty space, the importance of the perception difficulty space, the detected user perception movement, the counter-obstruction perception movement, and the level of the counter-obstruction perception movement.

29. An operation aptitude judgment device that judges an operation aptitude level indicating in how suitable condition a user is to perform a planned operation that should be carried out, the operation aptitude judgment device comprising:
a processing unit to execute a program; and
a memory to store the program which, when executed by the processor, performs
a process of making a perception difficulty space detection unit detect a perception difficulty space in which a perception object as an object that the user should perceive when the user performs the planned operation is difficult for the user to perceive based on vicinal object information acquired from a vicinal object detection device that detects a vicinal object existing in a vicinity of the user, and importance of the perception difficulty space;
a process of making a user perception movement detection unit detect a user perception movement, as a movement of the user when the user tries to perceive the perception object, a counter-obstruction perception movement as a user perception movement when the user tries to perceive a potential perception object as a perception object that can exist in the perception difficulty space, and a level of the counter-obstruction perception movement, based on user movement information acquired from a user movement detection device that detects a movement of the user; and
a process of calculating the operation aptitude level of the user based on the detected perception difficulty space, the importance of the perception difficulty space, the detected user perception movement, the counter-obstruction perception movement, and the level of the counter-obstruction perception movement.
